(19) United States
(12) Patent Application Publication
(10) Pub. No.: US 2024/0027395 A1
(43) Pub. Date: Jan. 25, 2024

(54) Graphene nanoribbon with nanopore-based signal detection and genetic sequencing technology

(71) Applicant: Acorn Genetics Inc., Chicago, IL (US)

(72) Inventors: Ana Cornell, Chicago, IL (US); Ryan Abbott, Vernon Hills, IL (US); Jake Turner, Scottsdale, AZ (US)

(21) Appl. No.: 18/224,971

(22) Filed: Jul. 21, 2023

Related U.S. Application Data
(60) Provisional application No. 63/391,154, filed on Jul. 21, 2022.

(51) Int. Cl. G01N 27/414; C12Q 1/6869

(52) U.S. Cl. CPC G01N 27/4145; C12Q 1/6869; G01N 27/4146; G01N 27/4148

(57) ABSTRACT

A silicon-based chip mounted on a graphene membrane that allows for more efficient DNA translocation measurements and nucleotide probing and analysis includes a Si substrate; a SiO₂ layer on top of the Si substrate; a SiNx layer; an electrode; and a graphene membrane on top of a surface of the SiNx layer.

### CROSS-REFERENCE TO RELATED APPLICATIONS

This application claims the benefit of priority to U.S. Provisional Application No. 63/391,154 filed Jul. 21, 2022, the entirety of which is incorporated by reference.

### BACKGROUND OF THE INVENTION

The present invention relates to a novel method of graphene nanoribbon (GNR) with nanopore (GNP) based genetic sequencing. More specifically, the present invention is directed to a graphene membrane mounted on a silicon based chip that enables more efficient DNA translocation measurements and nucleotide sequencing and analysis. It innovates in biotechnology and the biosciences, allowing institutions in academia, pioneers in industry, and everyday consumers to more easily obtain information on human genetic samples.

Sanger Sequencing, invented in 1975, was the culmination of a century and a half of biological inquiry initially begun in the early 1800s. In 1953, Watson and Crick had discovered the basic concepts and mechanisms for how the DNA underlying the genome is structured and functions. However, it was Frederick Sanger, as well as Allan Maxam and Walter Gilbert, who in 1975 each developed unique approaches to identifying the individual base pairs in a given sequence of DNA. With the Sanger and Maxam-Gilbert methods (using chain cleavage and chain termination, respectively) of genetic sequencing, scientists now possessed the capability to discern the series of distinct bases of DNA.

Only a quarter of a century later, the completion of the Human Genome Project in the early 21st century would rely heavily on an automated variation of Sanger Sequencing. Unfortunately, the team was hampered by limitations in the accuracy of base pair read-out and the length of fragments capable of being sequenced. These notable shortcomings, among others, prompted the development of “next generation sequencing,” also known as 2nd generation sequencing. This technology works by fragmenting genomes into small segments and then performing parallel sequencing on pre-selected, different sites of the DNA sample. As a result, it was well suited to observe thousands of samples and pinpoint one or a few key mutation sites over a short period of time, cutting the cost of sequencing by several orders of magnitude. The resulting high-throughput, accelerated speed, and improved accuracy enabled scientists to now sequence entire genomes, perform RNA sequencing and proteomic analyses, and even study organisms' microbiomes. But, while its research capabilities revolutionized the genetics space, many drawbacks remained. With only small segments able to be sequenced at once, understanding rare and more complex genetic diseases remained exceedingly arduous, as only the few research centers or hospitals possessing a wealth of genetic data could practically sequence in a diagnostic situation.

Furthermore, despite reducing the cost of sequencing by orders of magnitude, 2nd generation sequencers themselves remained incredibly expensive, often costing over $1M USD. These shortcomings spawned the desire for cheaper, longer read technologies, which are now known as 3rd generation sequencers. Contrary to the 2nd generation's high-throughput, short-read analysis of DNA, 3rd generation technology performs low-throughput, long-read analyses.

Nanopore sequencing is one of the most promising techniques in harnessing such technology. By measuring electrical perturbations in a fluid as long strands of nucleic acids quickly translocate the nanopore, it is inherently more rapid and scalable than its predecessors. The most commonly used substrates in this subfield are specialized proteins. While these are adequate for prototype 3rd generation sequencing, their short lifetime, mechanical and thermal instability, and proclivity to adsorb DNA strands have ultimately prevented initial nanopore technology from addressing the core flaws of the previous generations. Protein-based nanopore sequencers have reached the market, but their considerable drawbacks have impelled researchers to shift their focus to solid-state nanopore sequencing in search of more compelling solutions—most propitiously utilizing graphene as the substrate.

Unlike proteins, graphene monolayers are atomically thin, flexible, mechanically robust, and exceptionally conductive, in addition to being cost-effective and easily integrated with on-chip techniques. While this theoretically propels graphene-based nanopore sequencing to the top of the genetic sequencing ladder, fabrication difficulties, outsize signal noise, and control of DNA translocation have hampered its arrival.

Accordingly, there is a need for genetic sequencing technology capable of rapid, affordable long-read sequencing, thereby decentralizing access to genetic data, unlocking countless health benefits for all groups of people.

### BRIEF SUMMARY OF THE INVENTION

The present invention is directed towards resolving these concerns while simultaneously providing a novel design and fabrication process for the sequencing of nucleic acids using graphene nanopore technology. The following is intended to be a brief summary of the outlined invention, and the manner and order of the presentation is in no way limiting the overall invention's scope regarding fabrication and usage.

The methods and structures pertaining to this novel proposal center around a method of nucleic acid sequencing using both a solid-state nanopore, embedded in a graphene nanoribbon, and target sequence analysis using bimodal measurements of nucleic acid translocation events.

The invention relates to a machine (herein defined as the collected assembly of all components described) including but not limited to a microfluidic cassette, which may be composed of two thermoplastic-based half cells.

In one embodiment, said microfluidic cassette (also referred to as an ionic flow cell, microfluidic flow cell, or fluidic cell) contains a silicon (Si)/silicon nitride (SiNx) chip and a conductive, saline solution of determined ionic composition and concentration. The Si/SiNx chip may also include silver (Ag)/silver chloride (AgCl) electrodes for the cis and trans half cells of the microfluidic cassette. The Si/SiNx chip may be composed of a layer of Si onto which a layer of SiNx is deposited.

On the aforedescribed Si/SiNx chip, a graphene membrane, such as a monolayer graphene membrane, with a nanopore is deposited. In some embodiments, said graphene membrane is shaped into a graphene nanoribbon with a nanopore. It may also include an encapsulating material, such as Al₂O₃, to insulate the graphene.

In some embodiments, gold/chromium (Au/Cr) electrodes are deposited upon the aforedescribed monolayer graphene membrane. In other embodiments, the gold/chromium (Au/Cr) electrodes are deposited prior to graphene transfer onto the Si/SiNx chip. In alternative embodiments, these electrodes may also have an encapsulating layer, such as Al₂O₃.

Utilizing both sets of electrodes, a biased voltage is applied to facilitate ion movement and DNA translocation through the GNR with GNP. With experimental information including, but not limited to, ionic and electronic perturbations, a particular sequence may be derived for a segment of DNA.

In one embodiment of the present application, a chip includes a silicon (Si) substrate; a silicon nitride (SiNx) layer on the Si substrate; one or more electrodes; and a graphene sheet comprising a nanopore, the graphene sheet positioned atop the SiNx layer.

In some embodiments, the graphene sheet comprises a monolayer graphene nanoribbon. The nanopore may be centered in the monolayer graphene nanoribbon, and the monolayer graphene nanoribbon may be mounted to the one or more electrodes. The graphene sheet may be positioned atop the SiNx layer via Deep Ultraviolet (DUV) lithography or scanning thermal probe lithography. The nanopore may be approximately 1 nm in diameter.

The monolayer graphene may be configured for genetics sequencing. For example, the graphene nanoribbon including the nanopore may be used in a sequencer with particular optimized specifications to allow for nucleic acid probing and analysis.

In a further embodiment, a method comprises providing a chip including a silicon (Si) substrate; a silicon nitride (SiNx) layer on the Si substrate; one or more electrodes; and a graphene sheet comprising a nanopore, the graphene sheet positioned atop the SiNx layer; positioning the chip such that the one or more electrodes are parallel to a flow of nucleic acids; and measuring, through the one or more electrodes, an ionic current.

In some embodiments, the measuring step includes measuring a bimodal measurement of DNA or RNA nucleotide translocation by analyzing tunneling and ionic current simultaneously. The method may further include the step of providing a flow of an ionic fluid such as butylmethylimidazolium chloride (BMIM-Cl) solvent. The ionic fluid may be configured to stabilize nucleic acids for translocation and sequencing.

Any aspect disclosed and described herein may be combined with any other aspect or portion thereof described herein unless otherwise specified.

The above summary presents a high-level, simplified version of one or more embodiments in order to provide a basic understanding of said embodiments. It is in no way entirely encapsulating all possible variations or a detailed overview of contemplated embodiments—it further does not recognize any key elements. Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

Having briefly described some embodiments of the presented invention, they will now be illustrated to provide an example. The invention is not limited by the following figures (in regard to dimensionality, arrangement, or structure) and the following figures are not necessarily to scale.

FIG. 1 illustrates an annotated side elevational view of one example embodiment of the Si/SiNx chip with a GNR, GNP, and Au/Cr electrodes, according to the present application. Ag/AgCl electrodes are above and below the chip in this figure.

FIGS. 2A and 2B illustrate annotated side elevational views of the Si/SiNx chip of FIG. 1 with the Au/Cr electrodes encapsulated and the graphene and electrodes encapsulated, respectively. Ag/AgCl electrodes are above and below the chip in this figure.

FIG. 3 depicts an annotated top perspective view of the Si/SiNx chip of FIG. 1. The 60-nm diameter represents the pore in the SiNx membrane beneath the graphene layer.

FIG. 4 illustrates an annotated side elevational and top view of one example of the Si/SiNx chip of the present application in detail.

FIG. 5 illustrates a perspective view of an example of the Si/SiNx chip of the present invention. Here, a fragment of DNA can be seen translocating the nanopore of the Si/SiNx chip. Note that the graphene sheet and the DNA fragment are scaled up for greater detail of the structure.

FIG. 6 illustrates a perspective view of a further example of the present invention. Here, Au/Cr electrodes are encapsulated. Note that the graphene sheet is scaled up for greater detail of the structure. No DNA fragment is pictured.

FIG. 7 illustrates a perspective view of another example of the present invention. Here, while Au/Cr electrodes are not encapsulated, a GNR with GNP is depicted.

FIG. 8 illustrates a front elevational view of two polymer channels bonded with two thin films of polymer in between.

### DETAILED DESCRIPTION OF THE INVENTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefits and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

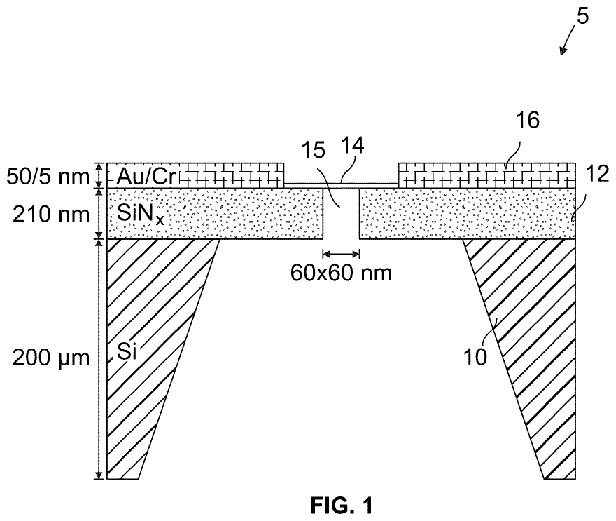

FIGS. 1-7 illustrate exemplary embodiments of a chip 5 of the present invention, including a silicon wafer 10 as the foundation. The wafer 10 is preferably a 4″ wafer to interface most effectively with equipment, but may be a wafer of any size or orientation (e.g. <1,0,0>) so long as necessary adjustments are made. To facilitate the fabrication process, P-doped Si is preferred to prevent retardation of wet Si etch processing. In some embodiments, double-side polished wafers will ensure measurements and modifications made to and encountered by the wafer 10 are precise. In some embodiments, the wafer 10 has a thickness of about 100 μm to about 500 μm, preferably about 200 μm, which is most adaptable for multiple options of fabrication.

A layer of SiNx 12 is deposited over the entire surface of the wafer 10 by Low Pressure Chemical Vapor Deposition (LPCVD) or another similar deposition technique, to introduce SiNx 12 to the Si surface 10. The SiNx layer 12 may have a thickness 210 nm or 305 nm, which will maximize the optical contrast between the substrate and the monolayer graphene to ease processing. A freestanding portion of the SiNx membrane 12, having a size ranging from 50×50 μm to 500×500 μm, is formed on an upper surface of the SiNx layer 12 using photolithography to lay down the pattern, Reactive Ion Etching (RIE) to remove the SiNx 12 on the backside, and wet KOH etching to create a well, preferably angled at 54.7°, in the silicon wafer 10 that leaves the remaining SiNx 12 freestanding. A minimized SiNx pore of 30-100 nm may be preferred in some embodiments.

In some embodiments, two sequential runs of RIE of half the recipe time result in superior and more controlled quality compared to a single recipe of full duration. In some embodiments, Deep Reactive Ion Etching (DRIE) may be used to etch partway through the silicon wafer 10 and finished by a wet etching technique, such as KOH etching, or, if the silicon wafer 10 is not <1,0,0> oriented, other (wet) etching techniques may be used to create a freestanding membrane. Wet etching should institute a non-horizontal protocol, whereby chip membranes are kept vertical whenever possible so they are not exposed to elevated pressures while submerged or under contact from liquids to prevent membrane fractures. The overall dimensions of the freestanding membrane should minimize the ratio of a length of the freestanding portion of the SiNx layer 12 to a thickness of the SiNx layer 12 to prevent fracturing during post-etching processes. Several hundred chip mounts would thus be fabricated on a single wafer.

Using one of Deep Ultraviolet (DUV) lithography, electron beam lithography (eBL), thermal scanning probe lithography (t-SPL), or Focused Ion Beam (FIB), a square or circular window 15 of about 20 nm to 100 nm in length or diameter, respectively, may be patterned into the center of the SiNx layer 12 of each chip 5. If necessary, the window 15 is etched with RIE where a resist is used to lay down a pattern, such as with polymethyl methacrylate (PMMA) in eBL). DUV lithography offers high throughput with longer process times and eBL offers precision but low throughput and high process times. Both are subject to the vagaries of wave fluctuations and behavior and require post-processing, including dry or wet etching. FIB grants pinpoint precision and control over each mount's window, but is difficult to automate and tune for drilling in each individual chip 5. It may be used for single chip mount milling if individual samples are required. Any combination of the above-mentioned methodologies as well as other suitable methodologies that are not listed above may be used as desired or required for manufacturing.

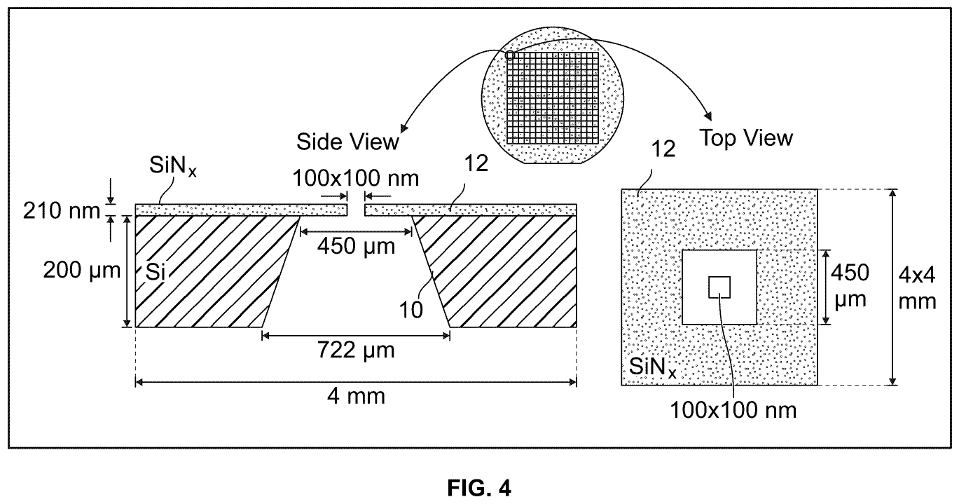

The technique of t-SPL may provide the precision benefits of eBL without the low throughput and long process times, as well as in situ evaluation of outcomes. The choice of instrument is subject to the concerns of the experimenter, but FIB and t-SPL appear most conducive to rapid prototyping. In further embodiments, dimensions of and methods of silicon chip coating and layer etching may differ but yield a result structurally similar or identical to the individual chip schematics presented in FIG. 4. Further, an additional SiO₂ layer of a determined thickness may be included for additional stability between the Si wafer 10 and the SiNx layer 12 through PECVD or an analogous technique.

This fabrication process is in no way limiting the scope of the invention; any analogous techniques in alternative workflow used to achieve similar outcomes as the above steps may be considered within the scope of the invention.

In various embodiments, the invention further includes a graphene monolayer 14. “Monolayer” herein is used to refer to a graphene membrane of one atom thickness. In some embodiments, said graphene may be grown on copper foil using high-temperature, high-pressure CVD; in others, it may be grown using mechanical exfoliation or electrochemical exfoliation in a solution of ammonium sulfate (or any similar ionic solution) and deionized water, vacuum filtration, and purification. Due to the handling difficulties and complexities associated with electrochemical exfoliation, it is a viable, but less efficient, method of graphene preparation. Mechanical exfoliation is a straightforward method providing the quickest graphene samples. Forms of CVD, followed by physical transfer methods such as those mediated by a support polymer, like PMMA, result in the most reproducible and reliable transfers of graphene to the substrate, particularly when pressure and heating can be used to facilitate graphene adhesion and preservation of its physical structure. In some embodiments, the monolayer graphene layer has a width of 0.345 nm. In other embodiments, the nanoribbon may have a 1:2.5 ratio.

This transfer may occur to individual 4×4 mm chips 5, by large-scale transfer to multiple chips 5 at once, or up to transfer onto an entire wafer. Experimental methods to verify adequate graphene transfer, and investigate the sculpting detailed below, may include, but are not limited to, Raman Spectroscopy, Scanning Electron Microscopy (SEM), optical microscopy, AFM, THz TDS, and charge mobility characterization.

In preferred embodiments, transfer of the graphene 14 to the Si/SiNx chip 5 may occur to create a graphene membrane 14 over the freestanding SiNx layer 12.

In a particular embodiment, wherein the graphene is grown using CVD, this transfer may be completed by spin-coating the graphene-support film substrate (the support film may be copper or a graphene-compatible polymer) in a layer of PMMA, etching away the copper support (if needed), transferring the graphene/PMMA stack to the Si/SiNx chip 5 through a wedging transfer or similar method, and dissolving away the PMMA. When possible, using a support polymer that is water-soluble in place of copper eliminates metallic contamination and may be preferred. Further, thermal treatment, as opposed to acetone-based removal, to remove the PMMA can leave fewer contaminants but requires significant experimental work to tune parameters for achieving this outcome.

In another embodiment, wherein the graphene is grown using electrochemical exfoliation, the transfer of graphene may be conducted using a Langmuir-Blodgett Trough. In said trough, exfoliated graphene (EG) and DMF-ethyl acetate solution may be dispersed at the air-water interface and compressed by the trough barriers, creating a graphene monolayer film 14 that may then be deposited onto the Si/SiNx chip 5 (and subsequently cleaned.) The aforedescribed description of the EG solution and process is not limited in its scope.

While the process of transfer following CVD and electrochemical exfoliation-derived graphene has been elaborated on in prior descriptions, they are in no way limiting of the scope of the invention which, in part, concerns the general transfer of grown graphene 14 onto the Si/SiNx chip 5.

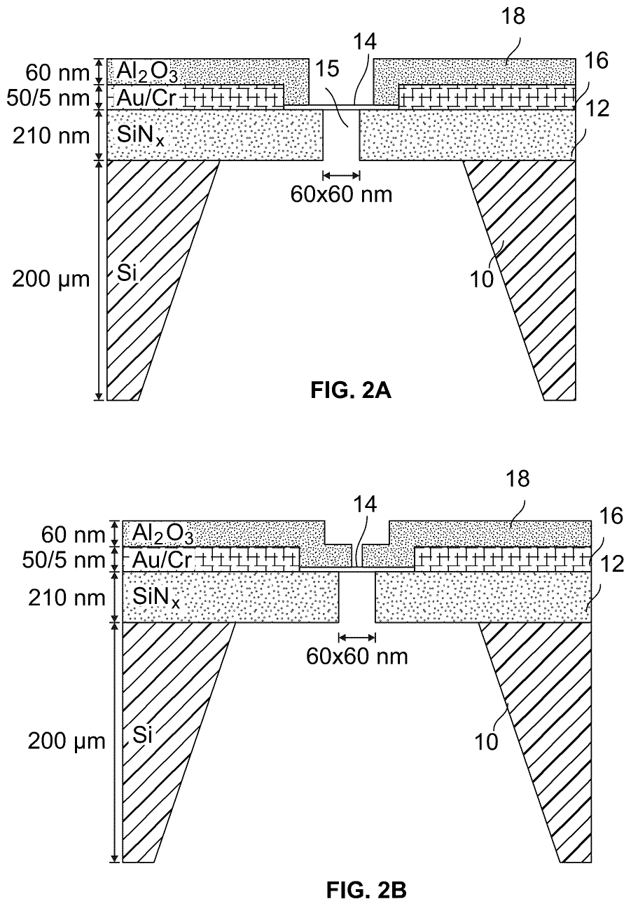

In some embodiments, a gold/chromium (Au/Cr) or silver (Ag)/silver chloride (AgCl) electrode 16 deposition, which may be completed using EBL, electron-beam evaporation, and lift-off, may follow graphene monolayer 14 transfer. In other embodiments, electrode 16 deposition may precede graphene 14 transfer. These processes are in no way limiting of the scope of possible techniques for electrode 16 deposition, and methods or materials producing analogous results will suffice so long as they produce electrodes 16 capable of acquiring graphene current and ionic current. Electrodes 16 may rest either directly over, under, or adjacent to the graphene monolayer 14 (so long as contact is made) and may be encapsulated, such as by a layer of Al₂O₃ 18 as shown in FIGS. 2A and 2B. One example of such encapsulation, which may also cover the graphene sheet 14 itself, is shown in concepts of some embodiments in FIG. 2B and FIG. 6. An example without encapsulation of one embodiment is given in FIG. 1.

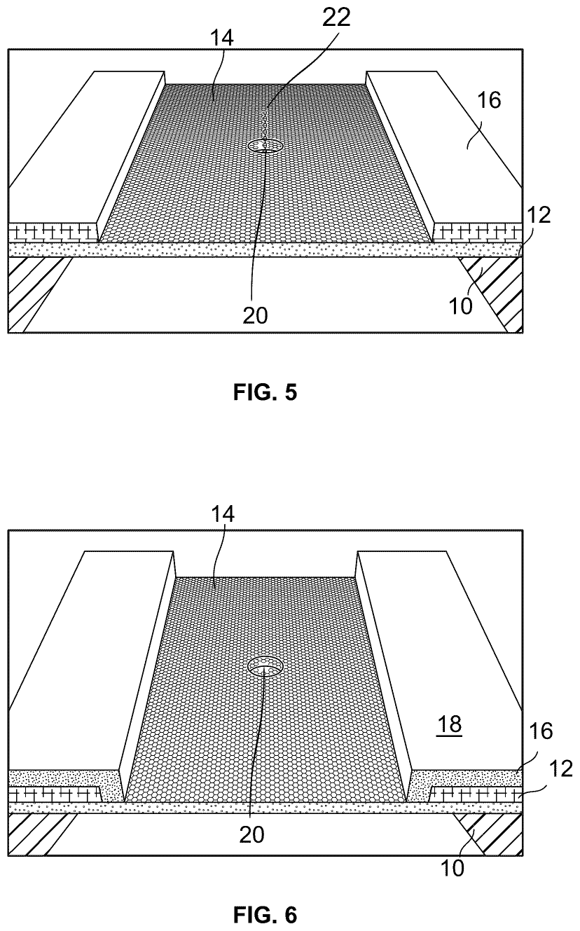

In some embodiments, a graphene nanoribbon may then be sculpted out of the existing graphene monolayer using, for example, eBL. A graphene nanopore 20 of less than about 5 nm, preferably less than about 3 nm, and most preferably about 1 nm, may be provided in the center of the ribbon or graphene sheet 14 as shown most clearly in FIGS. 5 and 6, depending on the embodiment. Scanning Transmission Electron Microscopy (STEM) or t-SPL may also be used to pattern these features, the latter technique of which requires the least post-processing. In some circumstances, high temperature STEM is preferred for creation of the nanopore 20 at minimized dimensions, and alternation between slow and fast scanning modes at 200-300 kV and elevated temperatures, such as 600 C or greater, will allow in situ sculpting and evaluation of the graphene membrane 14 at atomic precision. However, methods that use (photo)lithography or scanning probes may compete with or be superior to high temperature STEM in producing nanopores of desired geometry (here, ˜1 nm) if such techniques are precise enough to discern according dimensions. The chip 5 of FIGS. 1-4 includes the nanopore 20, although it is not shown.

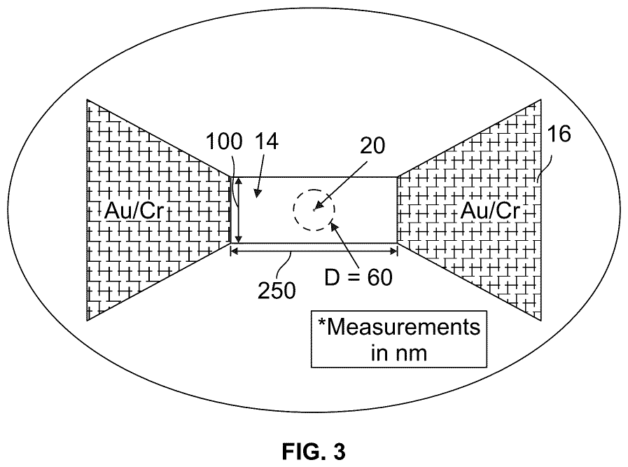

In particular, said graphene nanoribbon can be produced through a process of top-down high temperature eBL, wherein the original monolayer membrane may be etched to a width of about 100 nm and length of about 250 nm—in some embodiments, the desired region of the original graphene monolayer membrane 14 may be masked with nanowire protection, and the dimensions may be altered but retain its nanoribbon geometry; nanoribbons may be as small as ˜20 nm in width and ˜50 nm in length. This will depend in part on the geometry of the SiNx opening. FIG. 3 illustrates an example embodiment, although other dimensions and sizing may be utilized or preferred.

In some embodiments, to produce the best results, multiple nanoribbons or other graphene patterns may be laid onto a single chip 5, and the best structure(s) as determined by analysis through instruments and techniques given above and below would be selected for further processing on that chip.

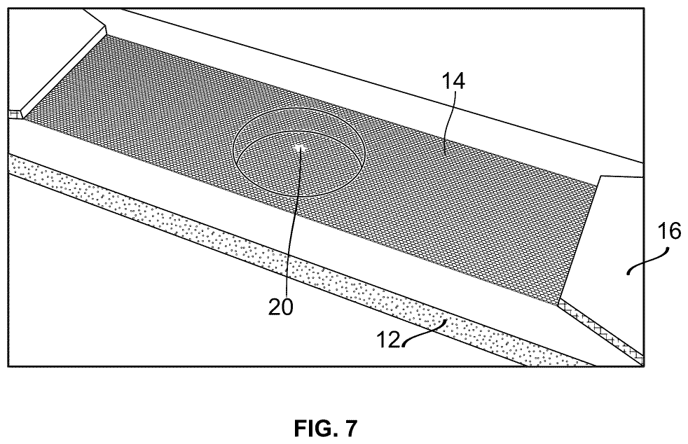

Following patterning of the graphene nanoribbon 14, testing of the quality of the nanoribbon through scanning tunneling microscopy (STM), Scanning Electron Microscope (SEM), and electrical conductance testing may be performed. Various embodiments of this invention may entail using a transmission electron microscope in high temperature (T≥600 C) STEM mode to perform electron-beam drilling and produce a 1 nm GNP within the nanoribbon. An example cross section of the chip 5 with the GNR 14 and the GNP 20 is depicted in FIG. 7.

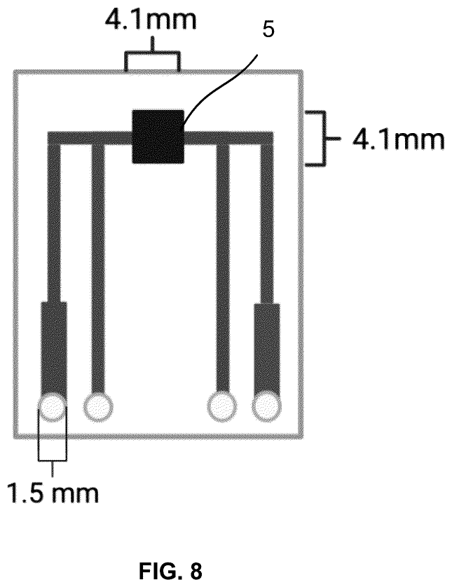

In various embodiments, the assembled Si/SiNx chip 5, GNR 14 with GNP 20, and Au/Cr electrodes 16 (collectively referred to as the “chip mounted graphene” for simplicity) is transferred to a constructed microfluidic cassette containing ionic fluid of determined concentration, pH, and volume, as shown schematically in FIG. 8. Said ionic fluid may be a butylmethylimidazolium chloride (BMIM-Cl) solvent, known to grant more controlled DNA translocation events, although other fluids may also be used. For example, potassium chloride (KCl) and/or cesium chloride (CsCl) may be used. By tuning fluidic parameters, such as concentration, pH, and temperature, better control of DNA structure, and therefore improved agency over translocation, may be obtained. For example, in some embodiments, a pH of 8-12.5 is preferred. In other embodiments, a molarity of 10 mM to 1 M is preferred.

In FIG. 8, the chip 5 is provided between two polymer channels bonded with two thin films of polymer in between. The chip is used to sequence DNA/RNA as it passes from one counter-current channel to the other.

In some embodiments, said microfluidic cassette, constructed from a range of materials including but not limited to thermoplastic polymers such as PDMS, may contain two distinct liquid reservoirs or “half-cells”, in between which may be positioned the chip-mounted graphene 14. Each Ag/AgCl electrode 16 is deposited in each half-cell of the microfluidic cassette, the chip 5 placed such that the electrodes 16 lie parallel to the flow of nucleic acids to be used for subsequent measurement of ionic current.

Measurement of experimental metrics includes, but is not limited to, electron tunneling current in plane to the graphene 14 acquired with the Au/Cr electrodes 16, in addition to the measurement of perpendicular ionic current with the Ag/AgCl electrodes 16. These measurements occur as nucleic acids translocate the nanopore 20, such as how DNA 22 is depicted in FIG. 5. As ions are occluded from passage through the nanopore 20 and electronic coupling occurs between the nucleic acids and graphene nanoribbon 14, characteristic decreases and increases in current, respectively, are acquired and analyzed to produce the translocated sequence of nucleic acids. For example, the use of the chip 5 may be used in a sequencer with particular optimized specifications to allow for nucleic acid probing and analysis.

The presently described chip may be used in the DNA or RNA sequencing of humans, viruses, bacteria, microbiomes, etc., although other uses may be envisioned by those of ordinary skill in the art. For example, the nanopore 20 may be used in a field hospital to quickly assess the nature of a patient's infection, bypassing the culturing that takes at least 24 hours and enabling physicians to utilize more specialized treatments more urgently as needed.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

We claim:

1. A chip comprising:
a silicon (Si) substrate;
a silicon nitride (SiNx) layer on the Si substrate;
one or more electrodes; and
a graphene sheet comprising a nanopore, the graphene sheet positioned atop the SiNx layer.

2. The chip of claim 1, wherein the graphene sheet comprises a monolayer graphene nanoribbon.

3. The chip of claim 2, wherein the nanopore is centered in the monolayer graphene nanoribbon, and the monolayer graphene nanoribbon is mounted to the one or more electrodes.

4. The chip of claim 2, wherein the monolayer graphene is configured for genetics sequencing.

5. The chip of claim 1, wherein the graphene sheet is positioned atop the SiNx layer via Deep Ultraviolet (DUV) lithography or scanning thermal probe lithography.

6. The chip of claim 1, wherein the nanopore is 1 nm.

7. A method comprising:
providing a chip comprising:
a silicon (Si) substrate;
a silicon nitride (SiNx) layer on the Si substrate;
one or more electrodes; and
a monolayer graphene nanoribbon comprising a nanopore, the monolayer graphene nanoribbon positioned atop the SiNx layer;
positioning the chip such that the one or more electrodes are parallel to a flow of nucleic acids; and
measuring, through the one or more electrodes, an ionic current.

8. The method of claim 7, wherein the measuring step includes measuring a bimodal measurement of DNA or RNA nucleotide translocation by analyzing tunneling and ionic current simultaneously.

9. The method of claim 7, further comprising the step of providing a flow of an ionic fluid.

10. The method of claim 9, wherein the ionic fluid is a butylmethylimidazolium chloride (BMIM-Cl) solvent.

11. The method of claim 9, wherein the ionic fluid is configured to stabilize nucleic acids for translocation and sequencing.
